(19) United States
(12) Patent Application Publication
(10) Pub. No.: US 2026/0207579 A1
(43) Pub. Date: Jul. 23, 2026

(54) VITAMIN B SUPPLEMENTATION TO TREAT PITUITARY PARS INTERMEDIA DYSFUNCTION (PPID) IN EQUIDS

(71) Applicant: MARS INCORPORATED, McLean, VA (US)

(72) Inventors: Patricia HARRIS, Melton Mowbray, Leicestershire (GB); David ALLAWAY, Melton Mowbray, Leicestershire (GB)

(73) Assignee: MARS INCORPORATED, McLean, VA (US)

(21) Appl. No.: 19/139,047

(22) PCT Filed: Dec. 15, 2023
(86) PCT No.: PCT/US2023/084440

§ 371 Date: Jun. 13, 2025

Related U.S. Application Data
(60) Provisional application No. 63/387,585, filed on Dec. 15, 2022.

(51) Int. Cl. A61K 31/455; A61K 31/198; A61K 31/355; A61K 31/714; A61K 35/20; A61K 36/87; A61P 5/08

(52) U.S. Cl. CPC A61K 31/455; A61K 31/198; A61K 31/355; A61K 31/714; A61K 35/20; A61K 36/87; A61P 5/08

(57) ABSTRACT

Compositions comprising niacin, a vitamin B3 related nicotinamide adenine dinucleotide precursors, or combinations thereof, are provided herein for use as a medicament, food supplement, or the like. Such compositions are provided to treat and prevent progression of pituitary pars intermedia dysfunction (PPID) in non-human animals, such as equids.

### CROSS REFERENCE TO RELATED APPLICATIONS

This application claims priority to U.S. Patent Application Ser. No. 63/387,585 filed on Dec. 15, 2022, which is incorporated herein by reference in its entirety.

### FIELD

The presently disclosed subject matter relates to dietary supplementation with niacin and vitamin B3-related nicotinamide adenine dinucleotide (NAD+) precursors to maintain health of equids with pituitary pars intermedia dysfunction (PPID), slow progression of PPID, reduce circulating concentrations of adrenocorticotropic hormone (ACTH), and maintain neuronal health of equids.

### BACKGROUND

Domesticated ponies and horses (Equus caballus) aged 15 years or older are classified as being senior and represent a substantial and apparently increasing proportion of the equine population (Jarvis et al., 2019). Aging is associated in equids, as with all animals, with an increased risk of certain conditions (Jarvis et al., 2019). The most prevalent endocrine disorder in aged equids being pituitary pars intermedia dysfunction (PPID) (Tatum et al., 2021).

PPID is characterized by hyperplasia, hypertrophy, and adenoma or microadenoma formation of the pituitary gland in the pars intermedia (PI) (McFarlane & Holbrook, 2008; Tatum et al., 2021). The chronic, progressive nature of PPID means that increasing age is a risk factor for its development, with a reported prevalence of over 20% in horses older than 15 years (Ireland and McGowan, 2018) and over 25% in horses older than 20 years old (Ballou et al., 2020). In the latter study, PPID was the second most common condition of aged horses, after osteoarthritis. Reported clinical signs of PPID are varied and include hypertrichosis/hirsutism, weight loss, abnormal fat redistribution, muscle wastage/atrophy, lethargy and depression, polyuria/polydipsia, and concurrent infectious diseases (Horn et al., 2019, Gallinelli et al., 2022). Laminitis, a painful key condition, which often results in the need for euthanasia, is also commonly reported in PPID cases. Laminitis is thought to occur predominantly in PPID cases with insulin dysregulation (ID) (Tadros et al., 2019).

Measurement of baseline plasma adrenocorticotropic hormone (ACTH) concentration is the most frequently used method to diagnose PPID worldwide (Horn & Bertin 2019). However, the thyrotropin-releasing hormone (TRH) stimulation test is a dynamic test that has been shown to be more sensitive for the diagnosis of PPID, especially in early stages of the disease when clinical signs are vague or when baseline ACTH concentrations are still within the reference range.

The pituitary gland comprises four different regions with different functions: the pars distalis, pars intermedia (PI), pars tuberalis (adenohypophysis) and pars nervosa (neurohypophysis). The PI has endocrine cells (melanotropes) which produce hormone precursor proteins—pro-opiomelancortin (POMC)-derived polypeptides. Following extensive tissue-specific cleavage, these yield in particular a-melanocyte-stimulating hormones (α-MSH), β-endorphin (β-end), corticotrophin-like intermediate lobe peptide (CLIP), and small quantities of adrenocorticotropic hormone (ACTH) (Horn et al., 2019). These melanotrophic endocrine cells are stimulated by nerve terminals of the hypothalamic-dopaminergic neurons resulting in the secretion of dopamine which acts through a negative feedback system to inhibit POMC transcription, as well as POMC peptide release (Hart et al., 2020). The pathophysiology of PPID is related to progressive degeneration of dopaminergic neurons from the hypothalamus, resulting in a loss of tonic inhibitory control over melanotrope cells of the pituitary pars intermedia (Van Der Kolk et al., 2004; and McFarlane, 2011). It has been suggested that oxidative stress may be involved in the pathophysiology (Tatum et al., 2020). In a healthy equid, mainly all ACTH is produced in the pars distalis; however, in a PPID equid, ACTH is excessively produced in the PI, as are multiple other small POMC molecules, which makes PI unique to equids.

Presently, the main pharmacologic treatment for PPID is the administration of pergolide mesylate, a dopamine receptor agonist previously used for treatment of Parkinson's disease, which has been shown to result in clinical improvement (Schott et al., 2019; Tatum et al., 2020; and Van Camp et al., 2004). Mitochondrial dysfunction and oxidative damage have been proposed as a key mechanism in many neurodegenerative diseases including Parkinson's Disease (Schondorg et al., 2018) and PPID (Clover et al., 2009; and Tatum 2020). Studies have suggested that supplementation with nicotinamide adenine dinucleotide (NAD+) precursors can be a viable clinical avenue to explore with Parkinson's disease (Schondorg et al., 2018). Although some recent work in mice using a proteasome inhibitor and lactacystin-induced model of Parkinson's disease has suggested that while there might be short term benefits, over the long term, at least nicotinamide riboside supplementation, may downregulate dopamine production (Turconi et al., 2021).

NAD+, an essential cofactor involved in many cellular processes, including energy metabolism and aging. Sirtuins (SIRTs) and poly-ADP-ribose polymerases (PARPs) are NAD+ dependent proteins and enzymes that consume NAD+ in their various reactions, depleting the cellular NAD+ pools. As such, there is a need to constantly replenish NAD+stores to keep these processes functioning within the cell. NAD+ is synthesized through various methods: the metabolism of dietary niacin in the Preiss-Handler pathway; through the conversion of the amino acid tryptophan via de novo synthesis; and through the salvage of nicotinamide (NAM).

Increasing the pool of NAD+ precursors and therefore increasing the potential concentration of NAD+ can help restore both mitochondrial dysfunction, decrease oxidative stress and age-related dopamine neuron loss in the horse and thereby improve the clinical signs associated with PPID, reduce circulating ACTH signs and potentially slow down progression in those animals with early signs of PPID. Achieving this through nutritional means, without the need for expensive pharmacological treatment with associated side effects such as inappetence, lethargy and diarrhea in around a ⅓^{rd} of cases (Tatum et al., 2020) or as an adjunct to such treatment in order to enable lower doses to reduce costs and risk of side-effects would be very beneficial.

Tryptophan supplementation has been associated with both excitatory and sedative effects in horses and high doses can case hemolytic anemia and symptoms of respiratory distress (Davis et al., 2017). Therefore, tryptophan supplementation is not a suitable route to increase NAD+ precursors in horses.

Equine feedstuffs contain varying amounts of naturally occurring niacin but in cereal grains, for example, the majority is in an unavailable bound form. The niacin content of green grasses is reported to be 37 mg (ranging from 13 to 57 mg/kg dry matter), although this can change considerably depending on the environmental stress, and the levels in timothy hay have been reported to be 24 mg/kg for example. Horses eating predominately green forage could ingest up to 1.5 g/day/500 kg horse (assuming maximum intake of ~5% BW/day, highest level and 100% availability) but could ingest less than 100 mg if on a restricted forage intake. Currently, the NRC 2007 has no recommendations for daily niacin intake in horses and INRA 1990 suggests a dietary requirement of 0.4 mg/kg BW/day for horses in intensive work (i.e., 200 mg/day) and a dietary intake of 12 mg niacin per kg dry matter intake as the daily requirement of adult horses in general (i.e., around 120 mg/day) (NRC 2007; and INRA 1990).

It has been suggested that nicotinic acid (NA) can be tolerated better than nicotinic acid amide (NRC 2007) due to the reported side effects. In other species, there have been reported adverse effects on fatty acids mobilization (Parker et al., 1997) as well as flushing, which has meant that NA is not considered a good possible therapeutic target for Parkinson's disease in human (Brady et al., 2020).

In horses, the potential effect of niacin as a putative ergogenic aid has been evaluated (Parker et al., 1997), however, there were no apparent benefits and no measured adverse effects of feeding 3 g/day to adult Thoroughbred horses likely around 500 kg for 6 weeks in exercising horses (Parker et al., 1997). Niacin status which is measured in through the ratio of NAD:NADP ratio in red blood cells was not affected by such levels of supplementation. However, this can have reflected the time of sampling post feeding (~12 hours) not being optimal as well as the dose used (Pollard et al., 2021, 22). In addition, it can suggest that under normal circumstances, basal dietary sources as well as production through hindgut fermentation (NRC 2007) can be adequate to maintain the ratio of NAD:NADP in red blood cells. Certainly, at the higher dose (5 g/day) used (Pollard et al., 2021) in horses, increases in plasma and urine concentrations of NAD+ precursors were seen but also low levels of potential metabolites suggesting some hepatotoxicity suggesting that a dose lower than this should be considered. More recent work by Pollard showed that feeding a lower dose (3 g/day: mean BW of ~500 kg)) of NA supplementation to mares increased the bioavailability of NAD+ precursors in the follicular fluid of mares despite no apparent increase in plasma concentrations (Pollard et al., 2022). NA would need to cross the blood brain barrier to reach the PI. In other species than the horse, exogenous NA has been shown to increase intracellular NAD+ levels in brain cells (Brady et al., 2020).

Currently, appropriate nutrition and management support is invaluable for PPID (Gallinelli et al., 2022), but these address the consequence of the condition and not the cause. There are no vitamin requirements specifically for PPID, although recommendations per age and activity level are suggested (Jarvis et al., 2019; and Gallinelli et al., 2022). There are changes in the structure of the microbiome (i.e., changes in diversity) associated with aging in horses (Dougal et al., 2014) and there is the potential in senior animals for disturbances in their microflora to reduce vitamin B production and absorption. A strong negative correlation between PPID and vitamin B12 in horses was reported (Siard-Altman et al., 2020), suggesting that some disturbance in vitamin B production can occur in PPID animals. Horses do not naturally ingest vitamin B12—it is formed through microbial activity in the gastrointestinal tract. Vitamin B3 status was not evaluated in this study but the studies finding might be a reflection of reduced vitamin B status in older and especially PPID horses. In addition, vitamin B12 supplementation in humans with dementia who had concurrent vitamin B12 deficiency has been reported to be beneficial (Ueno et al., 2022). PPID horses are often said to be depressed and lethargic and therefore vitamin B12 supplementation can provide additional advantages.

There remains a need in the art to maintain health of equids with pituitary pars intermedia dysfunction (PPID), slow progression of PPID, reduce circulating concentrations of adrenocorticotropic hormone (ACTH), and maintain neuronal health of equids. The present disclosure addresses this need with the finding that adequate Vitamin B3 additional supplementation alone or in combination with other nutritional support including additional vitamin B12, natural and synthetic antioxidants (to reduce any concomitant oxidative stress) as well as key amino acids to help combat the loss of muscle mass commonly associated with PPID (Erdody et al., 2023), therefore can be beneficial to the health and welfare of senior horses as well as those with PPID.

### SUMMARY

The purpose and advantages of the disclosed subject matter will be set forth in and are apparent from the description that follows, as well as will be learned by practice of the disclosed subject matter.

To achieve these and other advantages and in accordance with the purpose of the disclosed subject matter, as embodied and broadly described, the disclosed subject matter is directed to the use of niacin to maintain or support neuronal health of equids with PPID and/or aging equids.

The presently disclosed subject matter provides for a composition comprising niacin, a vitamin B3 related nicotinamide adenine dinucleotide precursors, or combinations thereof for use as a medicament.

In certain embodiments, the composition comprises niacin for use in treatment of pituitary pars intermedia dysfunction (PPID). In particular embodiments, the use is suitable for treating non-human animals. In certain embodiments, the non-human animal is an equid. In particular embodiments, the equid is a horse, pony, or donkey.

In certain embodiments, the equid weighs between about 350 kg to about 650 kg. In particular embodiments, the non-human animal is a senior animal. In certain embodiments, the non-human animal is about 15 years or older in age.

In certain embodiments, the composition is a supplement to the main diet of the non-human animal. In particular embodiments, the supplement is a powder, or crumbs, or solid form such as tablets, granules, or a liquid. In particular embodiments, the niacin is isolated from its original food source.

In particular embodiments, the composition can further comprise vitamin B12. In certain embodiments, vitamin B12 is isolated from its original food source.

In certain embodiments, the dosage of niacin is from about 1 to less than about 10 mg/kg body weight of the non-human animal per day. In particular embodiments, the dosage of vitamin B12 is from about 0.001 mg/kg to about 0.1 mg/kg of body weight of the non-human animal per day.

In certain embodiments, the composition further comprises amino acids. In particular embodiments, the amino acids are one or more amino acids selected from a group comprising lysine, methionine, threonine, and any branched chain amino acid such as leucine, valine, and isoleucine. In certain embodiments, the amino acid is lysine, the lysine is present in the composition in amounts of from about 2 g to about 10 g per day. In particular embodiments, the amino acid is methionine, the methionine is present in the composition in an amount of from 1 g to about 6 g dose per day. In certain embodiments, the amino acid is threonine, the threonine is present in the composition in an amount of from about 1 g to about 6 g dose per day. In particular embodiments, the amino acid is a branched chain amino acid or mixture of branched chain amino acids, the branched chain amino acid(s) are present in amounts of from about 5 g to about 25 g per day. In certain embodiments, the branched chain amino acid(s) is provided as whey protein, preferably the whey protein is provided in an amount of from about 2 g to about 15 g per day.

In particular embodiments, the composition further comprises one or more antioxidant(s). In certain embodiments, the one or more antioxidant(s) are natural or synthetic. In particular embodiments, the antioxidant is vitamin E. In certain embodiments, vitamin E is present in an amount of from about 50 units to about 2,000 units per day. In particular embodiments, the antioxidant is an antioxidant flavonoid, preferably quercetin. In certain embodiments, the quercetin is provided as a grape seed concentrate, preferably the grape seed concentrate is present in amounts of from about 0.25 g to about 10 g per day.

In particular embodiments, the composition of further comprises one or more ingredients selected from the group consisting of a binder, a flavorant, a carrier, wheat feed, rapeseed oil, a pharmaceutically acceptable excipient, a stabilizing agent, an anticaking agent, an emulsifier, and combinations thereof. In certain embodiments, the flavorant is a spearmint flavor or a rosemary extract. In particular embodiments, spearmint flavor is in the range of range of from about 1 to about 100 mg per day. In certain embodiments, the rosemary extract is present in the range of from about 5 mg to about 100 mg per day. In particular embodiments, the carrier is calcium carbonate present in a range of from about 1 mg to about 50 g per day. In certain embodiments, the wheat feed is present in a range of from about 100 mg to about 30 g dose per day. In particular embodiments, the rapeseed oil is present in a range of from about 150 mg to about 100 g dose per day.

In particular embodiments, the dosage is reduced over time in proportion to the easing of the severity of PPID symptoms. In certain embodiments, the dosage is increased as the animal ages.

The presently disclosed subject matter also provides for a composition comprising niacin, a vitamin B3 related nicotinamide adenine dinucleotide precursors, or combinations thereof, in a dose of from about 1 mg/kg to less than about 10 mg/kg body weight of a non-human animal per day.

In certain embodiments, the composition is a supplement to the main diet of the non-human animal. In particular embodiments, the supplement is a powder, or crumbs, or solid form such as tablets, granules, or a liquid.

In certain embodiments, the niacin is isolated from its original food source.

In particular embodiments, the composition further comprises vitamin B12. In certain embodiments, vitamin B12 is isolated from its original food source. In particular embodiments, the dosage of vitamin B 12 is between about 0.001 mg/kg to about 0.1 mg/kg of body weight of the non-human animal per day.

In certain embodiments, the composition further comprises amino acids. In particular embodiments, the amino acids are one or more amino acids selected from a group consisting of lysine, methionine, threonine, and any branched chain amino acid such as leucine, valine, and isoleucine, and combinations thereof. In certain embodiments, the amino acid is lysine, and the lysine is present in the composition in an amount of from about 2 g to about 10 g per day. In particular embodiments, the amino acid is methionine, and the methionine is present in the composition in an amount of from about 1 g to about 6 g per day. In certain embodiments, the amino acid is threonine, and the threonine is present in the composition in an amount of from about 1 g to about 6 g per day. In particular embodiments, the amino acid is a branched chain amino acid or mixture of branched chain amino acids, the branched chain amino acid(s) are present in amounts of from about 5 g to about 25 g per day. In particular embodiments, the branched chain amino acid(s) is provided as whey protein, and the whey protein is provided in an amount of from about 2 g to about 15 g per day.

In particular embodiments the composition further comprises one or more antioxidant(s). In certain embodiments, the one or more antioxidant(s) are natural or synthetic. In certain embodiments, the antioxidant is vitamin E. In particular embodiments, the vitamin E is present in an amount of from about 5 units to about 2,000 units per day. In certain embodiments, the antioxidant is an antioxidant flavonoid, preferably quercetin. In certain embodiments, quercetin is provided as a grape seed concentrate, preferably the grape seed concentrate is present in an amount of from about 0.25 g to about 5 g per day.

In certain embodiments, the composition further comprises one or more of the ingredients selected from a group consisting of a binder, a flavorant, a carrier, wheat feed, rapeseed oil, a pharmaceutically acceptable excipient, a stabilizing agent, an anticaking agent an emulsifier, and combinations thereof. In certain embodiments, the flavorant is a spearmint flavor or a rosemary extract. In certain embodiments, the spearmint flavor is in the range of range of from about 1 mg to about 100 mg per day. In certain embodiments, the rosemary extract is present in the range of from about 5 mg to about 100 mg per day. In certain embodiments, the carrier is calcium carbonate, preferably present in the range of from about 1 mg to about 50 g dose per day. In certain embodiments, the wheat feed is present in the range of from about 100 mg to about 30 g per day. In particular embodiments, the rapeseed oil is present in the range of from about 150 mg to about 100 g per day.

The presently disclosed subject matter further provides for the use of the compositions disclosed herein for the treatment of PPID in equid animals.

The presently disclosed subject matter also provides for a method for treating pituitary pars intermedia dysfunction (PPID) in an equid animal in need thereof, the method comprising administering a composition comprising niacin, a vitamin B3 related nicotinamide adenine dinucleotide precursors, or combinations thereof. In certain embodiments, the equid is a horse, pony, or donkey. In specific embodiments, the composition is a supplement to a main diet.

These and other features, aspects, and advantages of the disclosure will be apparent from a reading of the following detailed description. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### DETAILED DESCRIPTION

The present disclosure is based, in part, on the discovery that a dietary supplement with niacin and/or vitamin B3-related nicotinamide adenine dinucleotide (NAD+) improves clinical signs in equids with pituitary pars intermedia dysfunction (PPID). The dietary supplementation with niacin and/or vitamin B3-related nicotinamide adenine dinucleotide (NAD+) precursors to maintain health of equids with PPID, slow progression of PPID, reduce circulating concentrations of adrenocorticotropic hormone (ACTH), and maintain neuronal health of equids.

For clarity, but not by way of limitation, the detailed description of the presently disclosed subject matter is divided into the following subsections:
Definitions;
Equines;
Niacin-based Compositions; and
Methods of Treatment.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value.

The terms “embodiment,” “an embodiment,” “one embodiment,” “in various embodiments,” “certain embodiments,” “some embodiments,” “other embodiments,” “certain other embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with any other embodiment whether or not explicitly described.

The term “animal” as used in accordance with the present disclosure refers to equids, including horses, ponies, and donkeys.

As used herein, and as well-understood in the art, “treatment” is an approach for obtaining beneficial or desired results, including clinical results. For purposes of this subject matter, “treatment” means improving an existing medical condition, for example, by slowing down the disease progression, prolonging survival, reducing the risk of death, and/or providing a measurable improvement of disease parameters. “Treatment” can also mean prevention of disease and reducing the likelihood of developing disease.

As used herein, the terms “prevent,” “preventing” and “prevention” in the context of the administration of a composition to a subject refer to the prevention or inhibition of the onset and/or development of pituitary pars intermedia dysfunction (PPID).

As used herein, the term “pituitary pars intermedia dysfunction (PPID)” refers to an endocrine disorder that is neurodegenerative and common in aged equids (Tatum et al., 2021; and www.ceh.vetmed.ucdavis.edu/health-topics/pituitary-pars-intermedia-dysfunction-ppid).

As used herein, the term “niacin” generically encompasses and is alternatively known as nicotinic acid, nicotinamide, and related derivatives. Niacin is also known as vitamin B3 and can encompass vitamin B3-related nicotinamide adenine dinucleotide (NAD+) precursors (See www.ods.od.nih.gov/factsheets/Niacin-HealthProfessional). NAD+ precursors can encompass nicotinamide mononucleotide, nicotinamide riboside, nicotinic acid adenine dinucleotide, and nicotinic acid mononucleotide.

The term “food product” refers to a product or composition that is intended for consumption by, and provides certain nutritional benefits to a horse. In certain non-limiting embodiments, the horse is a member of the genus Equus caballus (e.g., a horse, a pony, or a donkey). A “food product” includes any food, feed, snack, food supplement, liquid, beverage, treat, toy (chewable and/or consumable toys), meal substitute, or meal replacement.

### Equine

The presently disclosed subject matter pertains to the population of animals under the genus Equus. This genus is part of the Equidae family, which includes numerous extant and extinct species. Species of the genus Equus include horses (Equus caballus), mules (Equus mulus), hinnies, wild horses (Equus ferus), mountain zebras (Equus zebra), African wild asses (Equus africanus), domestic donkey (Equus africanus asinus), Grévy's zebras (Equus grevyi), Asiatic wild asses (Equus hemionus), kiangs (Equus kiang), and plains zebras (Equus quagga).

In certain embodiments, the animal is of the species Equus caballus, which includes horses and ponies. The animal of the species Equus caballus may be of any age, gender or castration status, and so the invention can be used to assess (without limitation) foals, weanlings, yearlings, colts, fillies, mares, stallions, geldings or rigs. Any and all breeds of animals of the genus Equus, preferably of the species Equus caballus, can be assessed using methods of the invention.

In certain embodiments, the presently disclosed methods can be applied to non-human animals. In certain embodiments, the non-human animals are equids. In other specific embodiments, the equid is a horse, pony, or donkey.

### Niacin-Based Compositions

As is well-known in the art, “niacin,” also known as “Vitamin B3,” is a B vitamin that is consumed naturally from foods, as a dietary supplement, or as a food additive. “Niacin” can also refer to nicotinic acid, nicotinic acid amide, and/or their derivatives. Niacin is a water-soluble vitamin that is converted into NAD+ in the body. (See www.ods.od.nih.gov/factsheets/Niacin-HealthProfessional). According to the disclosed subject matter, niacin and/or other Vitamin B3-related nicotinamide adenine dinucleotide precursors are used herein as a medicament in the treatment of PPID.

In certain embodiments, the niacin is sourced naturally from food products. In alternative embodiments, the niacin is sourced via synthetic means.

In certain embodiments, niacin is formulated into a composition for oral consumption. In certain embodiments, niacin is administered in amounts ranging from about 0.5 g to less than about 5 g per 500 kg equid per day. In specific embodiments, niacin is administered in amounts ranging from about 0.5 g to about 4 g per 500 kg equid per day. In certain embodiments, niacin is administered in amounts from about 1 mg per kg to less than about 10 mg per kg of body weight per day, or from about 1 mg per kg to about 8 mg per kg of body weight per day.

In certain embodiments, vitamin B3 is administered on its own or together with vitamin B12. In particular embodiments, vitamin B3 is provided in amounts ranging from about 0.5 g to less than about 5 g per 500 kg equid per day, or from about 0.5 g to about 4 g per 500 kg equid, or from about 1 mg to less than about 10 mg per kg of body weight per day, or from about 1 mg to about 8 mg per kg of body weight per day on its own or together with vitamin B12 in amounts ranging from about 0.001 mg to about 0.1 mg per kg of body weight per day.

The composition can include a source of amino acids to help with muscle repair and development. In certain embodiments, the source of amino acids can include one or more of lysine, methionine, threonine, leucine, valine, and/or isoleucine. In certain embodiments, the source of amino acids can be whey protein. The amount of amino acid necessary for each equid is based on the body weight of the animal. By way of example, the average weight of an adult horse is from about 350 kg to about 650 kg. Based on this weight, in certain embodiments, the amino acids can be included in an amount from about 1 g to about 25 g, or from about 5 g to about 25 g, or from about 2 g to about 15 g, or from about 2 g to about 10 g, or from about 1 g to about 6 g. In certain embodiments, lysine can be present in an amount from about 2 g to about 10 g. In certain embodiments, methionine can be present in an amount from about 1 g to about 6 g. In certain embodiments, threonine can be present in an amount from about 1 g to about 6 g. In certain embodiments, leucine, valine, and/or isoleucine can be present in an amount from about 5 g to about 25 g. In certain embodiments, whey protein can be present in an amount from about 2 g to about 15 g. In certain embodiments, the amino acid is a branched chain amino acid or mixture of branched chain amino acids, the branched chain amino acid(s) are present in amounts of from about 5 g to about 25 g per day. The amounts of amino acids can be adjusted based on the body weight of the animal.

In certain embodiments, the composition can include natural or synthetic antioxidants. In certain embodiments, the antioxidants can include vitamin E and/or flavonoids. In certain embodiments, the flavonoid can be present in quercetin containing grape seed concentrate feedstuff. In certain embodiments, for example, the antioxidant can be included in an amount from about 5 IU to about 2,000 IU or from about 0.25 g to about 5 g for an average adult equid. As such amounts are based on the body weight of the animal, the amount used can be adjusted accordingly.

The composition can contain other ingredients which are used in an equine food, such as binders, flavorants, carriers, pharmaceutically acceptable excipients, stabilizing agents, anticaking agents, emulsifiers, palatability agents, fiber sources, fat sources, starch sources, and/or fillers. In certain nonlimiting embodiments, the flavorants can include one or more of spearmint flavor and/or rosemary extract. In certain nonlimiting embodiments, the carriers can include one or more of calcium carbonate, wheat feed and/or rapeseed oil or other such oils. In certain nonlimiting embodiments, molasses and/or a molasses and oil mixture can be included as a binder and/or palatability agent. In certain embodiments, the fiber sources can include one or more of grasses, sugar beet, soya hulls, and/or oats. In certain embodiments, the fat source can include one of more of corn oil, soya oil, processed canola oil, coconut oil, palm oil, and/or sunflower oil. In certain embodiments, the starch source is a cereal which can include one or more of a corn cereal, barley cereal, and/or oat cereal. In certain embodiments, the fillers can include one or more of oat feed and/or wheat feed.

In specific embodiments, spearmint flavor can be included in a range of from about 1 mg to about 200 mg, or from about 50 mg to about 150 mg, or from about 75 mg to about 125 mg. In certain embodiments, the spearmint is present in an amount of about 100 mg.

In specific embodiments, rosemary extract can be included in a range of from about 5 mg to about 100 mg, or from about 5 mg to about 50 mg, or from about 10 mg to about 50 mg, or from about 15 mg to about 45 mg, or from about 20 mg to about 30 mg. In certain embodiments, the rosemary extract is present in an amount of about 25 mg.

In specific embodiments, calcium carbonate can be included in a range of from about 50 mg to about 50 g, or from about 1 g to about 50 g, or from about 5 g to about 20 g. In certain embodiments, calcium carbonate is present in an amount of about 12 g.

In specific embodiments, wheat feed can be included in a range of from about 100 mg to about 30 g, or from about 150 mg to about 15 g, or from about 175 mg to about 1 g, or from about 100 mg to about 500 mg, or from about 150 mg to about 300 mg, or from about 200 mg to about 275 mg. In certain embodiments, wheat feed is present in an amount of about 230 mg to about 260 mg. In a specific embodiment, wheat feed is present in an amount of about 254 mg.

In specific embodiments, rapeseed oil can be included in a range of from about 150 mg to about 100 g, or from 150 mg to about 1 g, or from about 200 mg to about 10 g, or from about 150 mg to about 350 mg. In certain embodiments, rapeseed oil is present in an amount of about 250 mg.

### Methods of Treatment

The present disclosure provides for methods of supplementation with niacin and/or vitamin B3-related NAD+ precursors to improve clinical signs in equids with PPID, slow progression of PPID, treat/prevent PPID, and reduce circulating concentrations of ACTH.

The methods of the present disclosure also support metabolism in senior equids prone to hormonal challenges, maintain the health of PPID equids, support neuronal health in equids, maintain brain health of equids and senior equids, and support healthy aging in equids. These methods are achieved with the administration of a medicaments comprising niacin-based compositions as described herein.

The niacin-based composition can be administered to an equid of any age. In certain embodiments, the animal is a senior equid. In certain embodiments, the animal is an equid that is a horse, pony, or donkey and is aged at least about 15 years.

In certain embodiments, the niacin-based composition is administered to an equid that has been diagnosed with PPID in the early or late stages. In specific embodiments, the niacin-based composition is administered to slow the development of PPID.

The niacin-based composition can be administered orally in the form of a food, powder, crumbs, solid pellets, granules, tablets, or a liquid. In certain embodiments, the food can be a food supplement to a main diet or can be added to the complementary feed itself. In certain embodiments, the powder can be sprinkled on the food of the horse, pony, or donkey. In certain embodiments the powder or crumbs are in the form of a powder or solid.

In certain embodiments, the niacin-based composition can be administered daily. Dosage for an equid can be based on the weight of the animal. For example, the disclosed subject matter provides nonlimiting examples of dosage for an average adult equid weighing from about 350 kg to about 650 kg. A horse, pony, or donkey weighing less than about 350 kg can be administered a proportionally lower daily dose. A horse, pony, or donkey weighing more than about 650 kg can be administered a proportionally higher daily dose.

In certain embodiments, the niacin-based composition can be administered at an initial higher dose and reduced to a long-term lower maintenance or started at a lower dose and then increased as the equid ages. In certain embodiments, the dosage is reduced over time in proportion to the easing of the severity of PPID symptoms.

### EXAMPLES

The presently disclosed subject matter will be better understood by reference to the following Example, which is provided as exemplary of the presently disclosed subject matter, and not by way of limitation.

### Example 1: Benefits of a Diet With the Niacin-Based Composition

The present example compares the benefits of a diet supplemented with vitamin B3 or vitamin B3 and other ingredients to a normal diet.

Methods:

A horse is fed a diet containing between 1 mg/kg of body weight to 8 mg/kg of body weight a niacin-based composition daily for up to four weeks. The NAD+ precursors and metabolites in the blood and urine were measured to determine the optimal dosing.

The effect of the optimal dose is measured using 2 to 24 horses with PPID. The horses are divided into two, or preferably three, groups. One group is fed a normal diet, and one group is fed a normal diet supplemented with vitamin B3, and/or a group is fed a normal diet supplemented with vitamin B3 and a synergistic cocktail of ingredients including one or more ingredients such as antioxidants, other B vitamins such as vitamin B12, and amino acids. The horses are fed this diet for 1 to 12 months.

Data Collection:

Horses are administered TRH stimulation tests and their blood and optionally urine are collected before beginning the study, periodically during study, and at the conclusion of the study. The blood samples are used to measure ACTH and liver enzymes as well as optionally NAD+ precursors, dopamine, prolactin, and/or other POMCs such as α-MSH. Urine samples are used to optionally measure NAD+ precursors including indicating possible liver hepatotoxicity. Optionally, a clinical evaluation is used to monitor the muscle atrophy score, demeanor score, spontaneous blink rate, and/or body condition score.

* * *

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification. As one of ordinary skill in the art will readily appreciate from the disclosed subject matter of the presently disclosed subject matter, processes, machines, manufacture, compositions of matter, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

### REFERENCES

Ueno, A., Hamano, T., Enomoto, S., Shirafuj, N., Nagata, M., Kimura, H., Ikawa, M., Yamanura O., Yamanaka, D., Ito, T., Kimura, Y., Kuriyama, M., & Nakamoto, Y., (2022). “Influences of Vitamin B12 Supplementation on Cognition and Homocysteine in Patients with Vitamin B 12 Deficiency and Cognitive Impairment.” Nutrients 14, No. 7, 1494.
Ballou, M.E., Mueller, M.K., & Dowling-Guyer, S. (2020). Aging Equines: Understanding the Experience of Caring for a Geriatric Horse with a Chronic Condition. Journal of Equine Veterinary Science, 90, 102993.
Braidy, N., Villalva, M. D., & Eeden, S. V. (2020). Sobriety and Satiety: Is NAD+ the Answer? Antioxidants 9(5), 425.
Davis, B.P., Engle, T.E., Ransom, J.I. & Grandin, T. (2017). Preliminary evaluation on the effectiveness of varying doses of supplemental tryptophan as a calmative in horses. Applied Animal Behaviour Science, Volume 188, 2017, Pages 34-41.
Erdody et al. 2023 Muscle atrophy scores in a population of aged horses and ponies with and without PPID. GEES in press.
Glover, C. M., Miller, L. M., Dybdal, N. O., Lopez, A., Duckett, W. M., & McFarlane, D. (2009). Extrapituitary and pituitary pathological findings in horses with pituitary pars intermedia dysfunction: a retrospective study. Journal of equine veterinary science, 29(3), 146-153.
Dougal, K., de la Fuente, G., Harris, P.A., Girdwood, S.E., Pinloche, E., Geor, R.J., Nielsen, B.D., Schott II, H.C., Elzinga, S. and Newbold, C.J., (2014). Characterisation of the faecal bacterial community in adult and elderly horses fed a high fibre, high oil or high starch diet using 454 pyrosequencing. PloS one, 9(2), p.e87424.
Horn, R., & Bertin, F. R. (2019). Evaluation of combined testing to simultaneously diagnose pituitary pars intermedia dysfunction and insulin dysregulation in horses. Journal of veterinary internal medicine, 33(5), 2249-2256.Galinelli, N., Bailey, S.R., Bamford, N.J., & Harris, P.A. (2022). Nutritional considerations for the management of equine pituitary pars intermedia dysfunction Equine Veterinary Education in press
Hart, K. A., et al. (2020). Chapter 41—Endocrine and Metabolic Diseases. In B. P. Smith, D. C. Van Metre, & N. Pusterla, Large Animal Internal Medicine (Sixth Edition) 1352-1420. Mosby. www.doi.org/10.1016/B978-0-323-55445-9.00041-0.
Herbst, A.C., Johnson, M.G., Gammons, H., Reedy, S.E., Urschel, K.L., Harris, P.A. & Adams, A.A., (2022). Development and evaluation of a muscle atrophy scoring system (MASS) for horses. Journal of Equine Veterinary Science, p.103771
Horn, R., Bamford, N.J., Afonso, T., Sutherland, M., Buckerfield, J., Tan, R.H.H., Secombe, C.J., Stewart, A.J., & Bertin, F.R. (2019). Factors associated with survival, laminitis and insulin dysregulation in horses diagnosed with equine pituitary pars intermedia dysfunction. Equine Vet J, 51, 440-445.
INRA. 1990. L' Alimentation des Chevaux. W. Martin-Rosset (editor). INRA Publications, Paris. pp. 232.
Ireland, J.L. & McGowan, C. M. (2018). Epidemiology of pituitary pars intermedia dysfunction: A systematic literature review of clinical presentation, disease prevalence and risk factors. The Veterinary Journal, 235, 22-33.
Jarvis, N., Paradis, M. R. and Harris, P. (2019), Nutrition considerations for the aged horse. Equine Veterinary Education 31(2) 102-110.
McFarlane, D. (2011). Equine pituitary pars intermedia dysfunction. Vet Clin North Am Equine Pract, 27, 93-113.
McFarlane, D. & Holbrook, T.C. (2008). Cytokine dysregulation in aged horses and horses with pituitary pars intermedia dysfunction. 22, 436-442.
McGowan, T.W., Pinchbeck, G.P., & McGowan, C.M. (2013a). Evaluation of basal plasma a-melanocyte-stimulating hormone and adrenocorticotrophic hormone concentrations for the diagnosis of pituitary pars intermedia dysfunction from a population of aged horses. Equine Vet J, 45, 66-73.
McGowan, T.W., Pinchbeck, G.P., & McGowan, C.M. (2013b). Prevalence, risk factors and clinical signs predictive for equine pituitary pars intermedia dysfunction in aged horses. Equine Vet J, 45, 74-9.
NRC, N. R. C. (2007). Nutrient Requirements of Horses: Sixth Revised Edition, Washington, DC, The National Academies Press
Parker, A. L., L. M. Lawrence, S. Rokuroda, and L. K. Warren. (1997) “The effects of niacin supplementation on niacin status and exercise metabolism in horses.” In Proceedings of the 15th Symposium of the Equine Nutrition and Physiology Society, Fort Worth, Texas, p. 19.
Pollard, C. L., Gibb, Z., Swegen, A., Lawson, E. F., & Grupen, C. G. (2021). Nicotinic acid supplementation at a supraphysiological dose increases the bioavailability of NAD+ precursors in mares. Journal of Animal Physiology and Animal Nutrition, 105(6), 1154-1164.
Pollard, C.L., Gibb, Z., Clulow, J., Ruiz, A., Sheridan, A., Bahrami, M., Swegen, A. and Grupen, C.G., (2022). Supplemental Nicotinic Acid Elevates NAD+ Precursors in the Follicular Fluid of Mares. Animals, 12(11), p.1383.
Siard, M.H., Harris, P.A., Moffett-Krotky, A.D., Ireland, J.L., Betancourt, A., Barker V.D., McMurry K.E., Reedy, S.E., Adams, A.A. (2020) Relationships of inflamm-aging with nutritional status, body composition, age, and pituitary pars intermedia dysfunction in a senior horse population. Veterinary immunology and immunopathology 221: 110013
Schöndorf, D.C., Ivanyuk, D., Baden, P., Sanchez-Martinez, A., Cicco, S.D., Yu, C., Giunta, I., et al., (2018). The NAD+ Precursor Nicotinamide Riboside Rescues Mitochondrial Defects and Neuronal Loss in iPSC and Fly Models of Parkinson's Disease. Cell Reports, 23, 2976-2988.
Schott, H., Andrews, F., Durham, A., Frank, N., Hart, K., Kritchevsky, J., McFarlane, D., & Stewart, A.J. (2019). Recommendations for the diagnosis and treatment of pituitary pars intermedia dysfunction (PPID). Equine endocrinology group yearly report http://sites.tufts.edu/equineendogroup/.
Tadros, E.M., Fowlie, J.G., Refasl, K.R., Martenuik, J., & Schott, H.C., (2019). Association between hyperinsulinaemia and laminitis severity at the time of pituitary pars intermedia dysfunction diagnosis. Equine Vet J, 51, 52-56.
Tatum, R.C., McGowan, C.M., & Ireland, J.L. (2020). Efficacy of pergolide for the management of equine pituitary pars intermedia dysfunction: A systematic review. Vet J, 266, 105562.
Tatum, R. C., McGowan, C. M., Dean, R. S., & Ireland, J. L. (2021). Equine pituitary pars intermedia dysfunction: Identifying research priorities for diagnosis, treatment and prognosis through a priority setting partnership. PloS one, 16(1), e0244784.
Turconi, G., Alam, F., SenGupta, T., Pirnes-Karhu, S., Olfat, S., Schmidt, M. S., & Pirinen, E. (2021). Nicotinamide riboside alleviates Parkinson's disease symptoms but downregulates dopamine metabolism upon lactacystin-induced proteostasis failure. bioRxiv.
Camp, G.V., Flamez, A., Cosyns, B., Weytjens, C., Muyldermans, L., Van Zandijcke, M., De Sutter, J., Santens, P., Decoodt, P., Moerman, C., & Schoors, D. (2004) Treatment of Parkinson's disease with pergolide and relation to restrictive valvular heart disease. Lancet, 363, pp. 1179-1183.
Van Der Kolk, J.H., Heinrichs, M., Van Amerongen, J.D., Stooker, R.C., In de Wal, L.J. & van den Ingh, T. S. (2004). Evaluation of pituitary gland anatomy and histopathologic findings in clinically normal horses and horses and ponies with pituitary pars intermedia adenoma. Am J Vet Res, 65, 1701-7.

1. A composition comprising niacin, a vitamin B3 related nicotinamide adenine dinucleotide precursor, or combinations thereof for use as a medicament.

2-11. (canceled)

12. The composition of claim 1, further comprising vitamin B12.

13-16. (canceled)

17. The composition of claim 1, further comprising one or more amino acids selected from a group comprising lysine, methionine, threonine, and any branched chain amino acid such as leucine, valine, and isoleucine.

18. The composition of claim 17, wherein the amino acid is lysine present in the composition in amounts of from about 2 g to about 10 g per day.

19. The composition of claim 17, wherein the amino acid is methionine present in the composition in an amount of from 1 g to about 6 g dose per day.

20. The composition of claim 17, wherein the amino acid is threonine present in the composition in an amount of from about 1 g to about 6 g dose per day.

21. The composition of claim 17, wherein the amino acid is a branched chain amino acid or mixture of branched chain amino acids present in amounts of from about 5 g to about 25 g per day.

22. The composition of claim 21, wherein the branched chain amino acid(s) is provided as whey protein in an amount of from about 2 g to about 15 g per day.

23. The composition of claim 1, further comprising one or more antioxidant(s).

24. (canceled)

25. The composition of claim 23, wherein the antioxidant is vitamin E.

26. (canceled)

27. The composition of claim 23, wherein the antioxidant is quercetin.

28. The composition of claim 27, wherein the quercetin is provided as a grape seed concentrate present in amounts of from about 0.25 g to about 10 g per day.

29. The composition of claim 1, further comprising one or more ingredients selected from the group consisting of a binder, a flavorant, a carrier, wheat feed, rapeseed oil, a pharmaceutically acceptable excipient, a stabilizing agent, an anticaking agent, an emulsifier, and combinations thereof.

30. The composition of claim 29, wherein the flavorant is a spearmint flavor or a rosemary extract.

31. The composition of claim 30, wherein the spearmint flavor is in the range of range of from about 1 mg to about 100 mg per day.

32. The composition of claim 30, wherein the rosemary extract is present in the range of from about 5 mg to about 100 mg per day.

33. The composition of the claim 29, wherein the carrier is calcium carbonate present in a range of from about 1 mg to about 50 g per day.

34. The composition of claim 29, wherein the wheat feed is present in a range of from about 100 mg to about 30 g dose per day.

35. The composition of claim 29, wherein the rapeseed oil is present in a range of from about 150 mg to about 100 g dose per day.

36-65. (canceled)

66. A method of treating pituitary pars intermedia dysfunction (PPID) in an equid animal in need thereof, the method comprising administering a composition comprising niacin, a vitamin B3 related nicotinamide adenine dinucleotide precursor, or combinations thereof.

67-68. (canceled)
